(19) United States
(12) Patent Application Publication
(10) Pub. No.: US 2019/0160255 A1
(43) Pub. Date: May 30, 2019

(54) IMPROVED CATHETER

(71) Applicant: Shelverton Holdings Pty Ltd, Queensland (AU)

(72) Inventor: Caroline Luise Shelverton, Queensland (AU)

(21) Appl. No.: 16/321,362

(22) PCT Filed: Jul. 28, 2017
(86) PCT No.: PCT/AU2017/050786

§ 371 Date: Jan. 28, 2019

(30) Foreign Application Priority Data
Jul. 29, 2016 (AU) 2016903005
Apr. 21, 2017 (AU) 2017901464

(51) Int. Cl. A61M 25/00; A61M 1/00

(52) U.S. Cl. CPC A61M 25/007; A61M 1/008; A61M 25/0021; A61M 2025/006; A61M 2025/0056; A61M 25/0043

(57) ABSTRACT

The present disclosure relates to a catheter for insertion into a vessel of a patient, the catheter including an elongate body extending between a proximal end for attachment to a hub and a distal end at a tip portion thereof, the body having a circumferential wall and including a first portion that extends a first length from the proximal end and defines an enclosed section of the wall and a second portion that extends a second length from an end of the first portion towards the tip portion, the second portion defining an at least partially open section of the wall having a plurality of openings arranged to permit fluid flow into and out of the second portion of the body.

### PRIORITY DOCUMENTS

The present application claims priority from Australian Provisional Application No. 2016903005 titled “IMPROVED CATHETER” filed on 29 Jul. 2016 and Australian Provisional Application No. 2017901464 titled “IMPROVED CATHETER” filed on 21 Apr. 2017, the contents of which are hereby incorporated by reference in their entirety.

### BACKGROUND OF THE INVENTION

The present invention relates to catheters, and in one example to a peripheral intra-venous catheter (PIVC) or midline catheter used for administering intravenous fluids, blood products and medications to a patient or aspirating blood or fluid for sampling. In another example, the invention relates to an arterial catheter used for haemodynamic monitoring and aspiration for blood sampling.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

A peripheral intra-venous catheter (PIVC) or arterial catheter or line (‘a-line’) is a vital tool in the delivery of patient care within a hospital or care facility. PIVCs are used by 70% of patients admitted to hospitals to administer intra-venous (IV) medicaments, fluids, nutrition and blood products and can be life saving. Arterial catheters are commonly used for haemodynamic monitoring and aspiration for blood sampling.

Traditional PIVCs and a-lines include a hollow tube typically made from a polymer such as polyurethane that is peripherally inserted into a vessel of a patient. The catheter tube typically has a smooth hydrophobic surface and a single lumen that terminates in a tip portion having a fluid outlet.

Currently, up to 30-40% of PIVCs fail for reasons due to occlusion, phlebitis, infiltration, infection and dislodgement while up to 35% of peripheral arterial lines fail for reasons due to fibrin formation and micro-aggregation of blood components which leads to thrombus. An essential requirement of a-lines is patency to allow adequate haemodynamic monitoring.

An example of a prior art catheter 100 (PIVC or a-line) for insertion into a vessel 10 of a patient is illustrated in FIG. 1, in which the catheter 100 is shown in situ within the vessel 10 (which may be a vein or artery).

In this example, the catheter 100 includes an open tube 110 extending between a proximate end 111 attached to a hub 120 and a distal end 112 that defines an opening that permits fluid 2 to flow into/out of the tube 110.

The tube 110 defines a single lumen and includes a generally tapered tip portion 113 that terminates at the opening of the distal end 112. The catheter 100 can be used to administer a fluid into the vessel 10 of the patient or for aspirating or drawing blood for sampling.

The drawbacks of this traditional prior art catheter shall now be described in further detail.

A first problem resides in the potential for interference between the catheter tip 113 and the vessel wall 12. Typically, in use, the catheter tip 113 will contact or drag against portion A of the inner layer of the vessel wall 12 known as the tunica intima 12a. This may cause damage to the vessel wall including erosion of the tunica intima 12a through friction which can in turn lead to phlebitis or infiltration. Phlebitis relates to inflammation or irritation of the walls of the vessel whilst infiltration concerns the potential for the catheter to penetrate or pierce through the vessel wall entirely.

Phlebitis may also result from the fact that the fluid outflow is typically concentrated on a small area of the vessel wall. Irritants such as IV medications that continuously exit the catheter onto the tunica intima in the same position may lead to inflammation.

Traditional single lumen catheters of the type shown in FIG. 1 are also prone to thrombotic occlusion whereby a thrombus forms within, surrounding or at the tip of the catheter, thereby blocking fluid flow into/out of the catheter. Occlusion may also result during aspiration of blood or fluids using a traditional single lumen catheter. The combination of proximity of the catheter tip to the tunica intima and negative pressure whilst aspirating may create a suction-like effect leading to a ball-valve occlusion at the catheter tip.

Peripheral arterial catheters used for example in haemodynamic monitoring may also become partially occluded by fibrin formation and micro thrombi.

Prior art catheters also generally have a smooth hydrophobic surface which attracts biofilm that can lead to microbial infection, particularly if the biofilm is dislodged and enters the bloodstream.

A final drawback of existing PIVC and arterial catheters relates to the shear force exerted on the vessel walls which may lead to wall damage or irritation. Typically, as a fluid is injected into a catheter from a pre-filled syringe the inflow pressure is constant. However, the lumen of a traditional catheter typically narrows towards the tip which increases the pressure and shear force exerted on the vessel wall as the fluid exits the catheter.

It is against this background, and the problems and difficulties associated therewith, that the present invention has been developed.

### SUMMARY OF THE PRESENT INVENTION

In one broad form, an aspect of the present invention seeks to provide a catheter for insertion into a vessel of a patient, the catheter including an elongate body extending between a proximal end for attachment to a hub and a distal end at a tip portion thereof, the body having a circumferential wall and including a first portion that extends a first length from the proximal end and defines an enclosed section of the wall and a second portion that extends a second length from an end of the first portion towards the tip portion, the second portion defining an at least partially open section of the wall having a plurality of openings arranged to permit fluid flow into and out of the second portion of the body.

In one embodiment, the plurality of openings are arranged in a matrix or web-like formation.

In one embodiment, the plurality of openings are elliptically shaped and extend in a direction of elongation of the body.

In one embodiment, the plurality of openings include:

a) openings having substantially the same length; and,

b) openings having varying length.

In one embodiment, the plurality of openings include a first arrangement of openings on a first side of the second portion and a second arrangement of openings on an opposing second side of the second portion, the openings on the first side being larger than the openings on the second side.

In one embodiment, a diameter of the openings on the first side is between 1.5 and 2 times a diameter of the openings on the second side.

In one embodiment, the ratio of the first length to the second length is approximately in the range 0.5 to 2.

In one embodiment, the plurality of openings reduce the surface area of the second portion of the body by at least one of:

a) between 30 and 40%;

b) between 40 and 50%;

c) between 50 and 60%;

d) between 60 and 70%; and,

e) between 70 and 80%.

In one embodiment, the second portion includes the tip portion.

In one embodiment, the circumferential wall defines an inner surface and an outer surface and wherein at least a portion of at least one of the inner and outer surface is textured or roughened so as to reduce biofilm adhesion to the body in use.

In one embodiment, a surface of the tip portion is textured or roughened so as to reduce biofilm adhesion to the body in use.

In one embodiment, an antibacterial coating is applied to at least a portion of the circumferential wall to reduce biofilm adhesion.

In one embodiment, the coating is an oil-infused polydimethylsiloxane (iPDMS).

In one embodiment, in use, when the catheter is inserted into the vessel and fluid outflow is at a constant rate, forces acting on the body are such that a spacing is maintained between the body and an internal wall of the vessel.

In one embodiment, at least a portion of the body is substantially centralized within the vessel.

In one embodiment, the catheter is a peripheral intra-venous catheter (PIVC).

In one embodiment, the catheter is an arterial catheter.

In another broad form, an aspect of the present invention seeks to provide a catheter for insertion into a vessel of a patient, the catheter including an elongate body extending between a proximal end for attachment to a hub and a distal end at a tip portion thereof, the body having a circumferential wall and including a portion defining an at least partially open section of the wall having a plurality of openings arranged to permit outflow of fluid from the body into the vessel such that in use, when the catheter is inserted into the vessel and fluid outflow is at a constant rate, forces acting on the body are such that a spacing is maintained between the body and an internal wall of the vessel.

In one embodiment, the plurality of openings are arranged in a matrix or web-like formation.

In one embodiment, at least a portion of the body is textured or roughened so as to reduce biofilm adhesion.

In a further broad form, an aspect the present invention seeks to provide a catheter assembly, including:
a) a catheter tube for insertion into a vessel of a patient, the catheter tube having an elongate body extending between a proximal end and a distal end at a tip portion thereof, the body having a circumferential wall and including a first portion that extends a first length from the proximal end and defines an enclosed section of the wall and a second portion that extends a second length from an end of the first portion towards the tip portion, the second portion defining an at least partially open section of the wall having a plurality of openings arranged to permit fluid flow into and out of the second portion of the body;
b) a hub attached to the proximal end of the catheter tube; and,
c) an introducer needle inserted through the catheter tube having an edge that projects beyond the distal end of the tube for penetrating a wall of the vessel.

In one embodiment, the catheter assembly further includes a guidewire that extends through a lumen of the introducer needle for use in guiding the catheter tube into the vessel.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

Non-limiting examples of the present invention will now be described with reference to the accompanying drawings, in which:

FIG. 1 is a schematic side view of a prior art catheter located in situ within a vessel of a patient;

FIG. 2 is a schematic side view of a first example of a catheter for insertion into a vessel of a patient;

FIG. 3 is a schematic side view of a second example of a catheter for insertion into a vessel of a patient;

FIG. 4 is a schematic side view of a third example of a catheter for insertion into a vessel of a patient

FIG. 5A is a schematic side view of a fourth example of a catheter for insertion into a vessel of a patient;

FIG. 5B is a schematic sectional view taken through line A-A of the catheter of FIG. 5A;

FIG. 6A is a schematic side view of the catheter of FIG. 5A inserted into a vessel and used to deliver a fluid into the bloodstream;

FIG. 6B is a schematic sectional view taken through line B-B of FIG. 6A showing fluid outflow from the catheter into the vessel;

FIG. 7 is a schematic side view of the catheter of FIG. 5A inserted into the vessel and used to draw a sample of blood into a syringe;

FIG. 8A is a schematic side view of an example of a first stage of inserting the catheter of FIG. 5A into the vessel using a guidewire;

FIG. 8B is a schematic side view of an example of a second stage of inserting the catheter of FIG. 5A into the vessel using a guidewire;

FIG. 9 is a schematic side view of a fifth example of a catheter for insertion into a vessel of a patient; and,

FIG. 10 is a schematic side view of a sixth example of a catheter for insertion into a vessel of a patient.

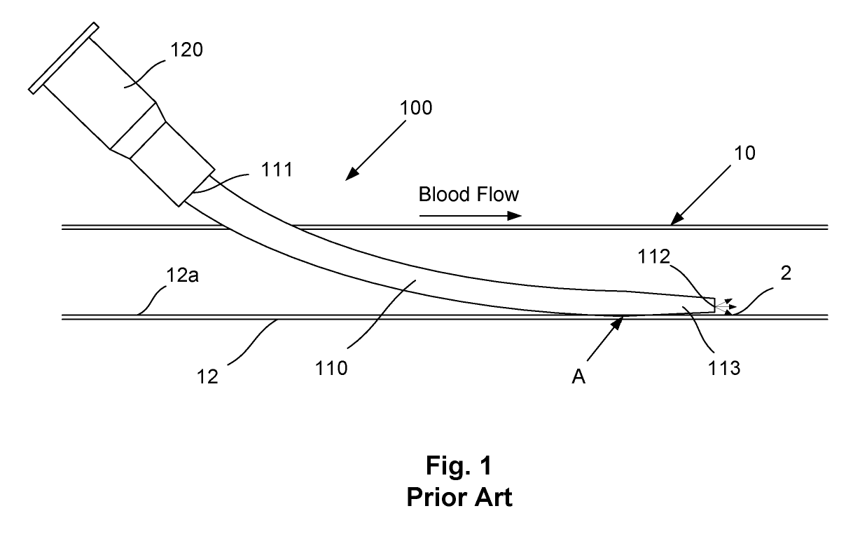

### DETAILED DESCRIPTION OF THE PREFERRED EMBODIMENTS

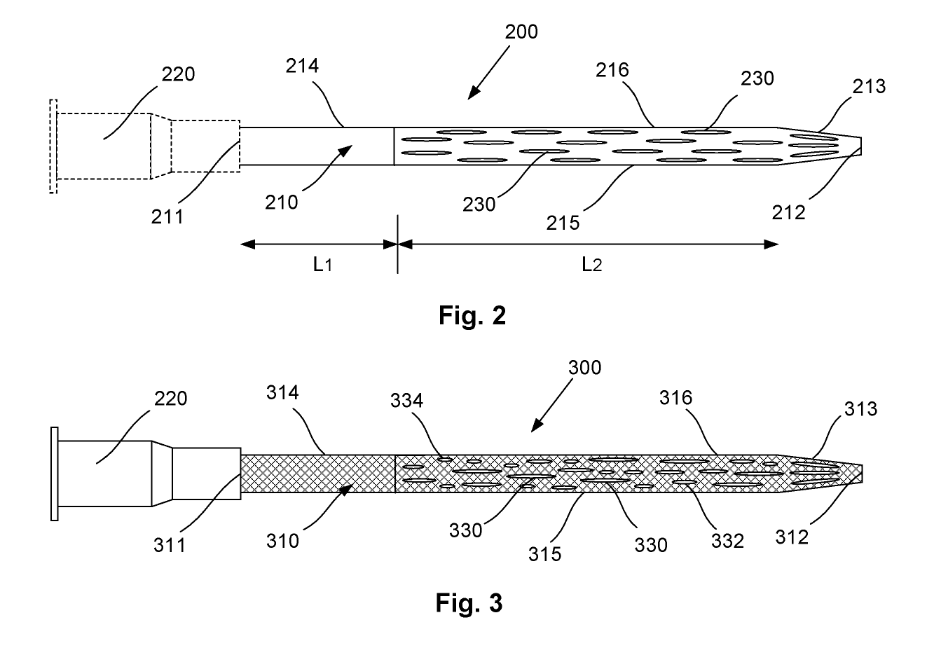

An example of a catheter 200 for insertion into a vessel of a patient will now be described with reference to FIG. 2. It is to be understood that as used herein, the term catheter refers to a range of medical devices for insertion into vessels of patients including peripheral intra-venous catheters (PIVC) or midline catheters used for delivering intra-venous (IV) fluids to a patient such as medicaments, nutrition and the like or aspirating blood or fluids for sampling, arterial catheters used for aspiration of blood and haemodynamic monitoring, and other types of catheters such as a renal arteriovenous fistula (AVF) catheters, central venous catheters, or the like.

A midline catheter (typically 7.5 cm to 20 cm in length) is inserted near the antecubital fossa into the basilic, cephalic, or brachial veins. The catheter tip is advanced no further than the distal axillary vein in the upper arm.

In this example, the catheter 200 includes an elongate body 210 extending between a proximal end 211 for attachment to a hub 220 and a distal end 212 at a tip portion 213 thereof, the body 210 having a circumferential wall 215 and including a first portion 214 that extends a first length L₁ from the proximal end 211 and defines an enclosed section of the wall and a second portion 216 that extends a second length L₂ from an end of the first portion 214 towards the tip portion 213, the second portion 216 defining an at least partially open section of the wall 215 having a plurality of openings 230 arranged to permit fluid flow into and out of the second portion 216 of the body 210.

The above described arrangement provides a number of advantages.

By providing a plurality of openings in the circumferential wall in a portion of the catheter, fluid is allowed to enter/exit the catheter at multiple positions around the circumference in addition to the fluid outlet provided at the distal end of the catheter. Fluid outflow is therefore not concentrated at a single point on the tunica intima of the vessel wall thereby minimizing the likelihood of inflammation or phlebitis. Furthermore, as fluid is able to exit the catheter around the circumference of the body, in at least some examples the forces acting on the catheter body may result in the catheter being substantially centralized within the vessel ensuring that the body is spaced from the tunica intima to thereby prevent drag and minimize the likelihood of tissue damage or erosion and infiltration of the vessel wall.

Thrombotic occlusion at the tip of the catheter is also eliminated due to the plurality of alternative fluid entry/exit points in the body of the catheter in addition to the fluid outlet at the tip. The above described arrangement is therefore particularly suitable for use in conjunction with keep vein open (TKVO) protocols and/or intermittent flushing. Ball-valve occlusion whilst aspirating is also eliminated as fluid is able to enter the catheter via the plurality of openings while bypassing the fluid inlet/outlet at the tip.

The plurality of openings in the body of the catheter further act to reduce the surface area of the body which reduces the area upon which biofilm is able to form. Furthermore, the openings in the body provide an alternative conduit for fluid flow leading to a reduction in the outflow shear force exerted on the vessel walls whilst also reducing the risk of dislodgement of biofilm into the bloodstream. A reduction in shear force is beneficial as it limits irritation of the vessel wall and any potential inflammatory response.

A further benefit resides in the reduction of outflow or inflow resistance as a result of providing multiple fluid entry/exit points in the body of the catheter, which improves transmission of pulsatile arterial waveform.

A number of further features shall now be described.

In one example, the plurality of openings are arranged in a matrix or web-like formation. In such an arrangement, the circumferential wall of the second portion of the catheter body may be formed in a web, mesh or lattice like structure that provides high rigidity ensuring the structural integrity of the wall whilst maximizing the reduction in surface area of the wall which leads to reduced biofilm build-up. The matrix structure may have a wave-like pattern that regularly repeats promoting uniform fluid inflow/outflow about the circumference of the catheter body. The matrix or web structure is typically a fine mesh to ensure that an introducer needle cannot easily protrude through any of the plurality of openings.

In another example, the plurality of openings are elliptically shaped and extend in a direction of elongation of the body. The plurality of openings may include openings having substantially the same length or openings having varying length. The elliptical openings may help to reduce the surface area of the second portion of the body.

In other examples, the plurality of openings may have different sizes depending on their position on the second portion. For instance, the plurality of openings may include a first arrangement of openings on a first side of the second portion and a second arrangement of openings on an opposing second side of the second portion, the openings on the first side being larger than the openings on the second side. In some examples, a diameter of the openings on the first side may be between 1.5 and 2 times a diameter of the openings on the second side.

The plurality of openings may reduce the surface area of the second portion of the body by between 30-40%, 40-50%, 50-60%, 60-70% and 70-80%. More specifically, the surface area may be reduced by between 30-35%, 35-40%, 40-45%, 45-50%, 50-55%, 55-60%, 60-65%, 65-70%, 70-75% and 75-80%.

In one example, the ratio of the first length to the second length is approximately in the range 0.5 to 2. In other words, the first length is approximately between one third and two thirds of the total length of the catheter and the second length is approximately two thirds to one third of the total length. This configuration allows for variation in the first length to suit variable patient build and vessel depth. In addition, this also ensures that the plurality of openings are provided over a substantial portion of the body whilst maintaining an enclosed solid portion that extends from the proximal end of the body so that backtracking of fluid out of the catheter is prevented.

In one example, the second portion includes the tip portion, however this is not essential and the tip portion may have an enclosed circumferential wall or alternatively may have separate openings to the arrangement of openings provided in the second portion.

Typically, the circumferential wall defines an inner surface and an outer surface and at least a portion of at least one of the inner and outer surface is textured or roughened so as to reduce biofilm adhesion to the body in use. Any suitable form of micro-texture or surface roughness may be applied so as to reduce the smoothness of the polymer surface of the body. In some examples, a surface of the tip portion may be textured or roughened in a similar manner.

In some examples, an antibacterial coating is applied to at least a portion of the circumferential wall to reduce biofilm adhesion. A suitable coating may be an an oil-infused polydimethylsiloxane (iPDMS).

Typically in use, when the catheter is inserted into the vessel and fluid outflow is at a constant rate, forces acting on the body are such that a spacing is maintained between the body and an internal wall of the vessel. In one example, at least a portion of the body is substantially centralized within the vessel. In effect, a ‘fluid cushion’ is provided between the catheter body and the vessel wall thereby preventing the catheter tip from dragging on the tunica intima and causing damage thereto. Tissue erosion and irritation is thereby avoided reducing the likelihood of phlebitis and infiltration.

In another broad form there is provided a catheter for insertion into a vessel of a patient, the catheter including an elongate body extending between a proximal end for attachment to a hub and a distal end at a tip portion thereof, the body having a circumferential wall and including a portion defining an at least partially open section of the wall having a plurality of openings arranged to permit outflow of fluid from the body into the vessel such that in use, when the catheter is inserted into the vessel and fluid outflow is at a constant rate, forces acting on the body are such that a spacing is maintained between the body and an internal wall of the vessel.

In yet a further broad form, there is provided a catheter assembly, including a catheter tube for insertion into a vessel of a patient, the catheter tube having an elongate body extending between a proximal end and a distal end at a tip portion thereof, the body having a circumferential wall and including a first portion that extends a first length from the proximal end and defines an enclosed section of the wall and a second portion that extends a second length from an end of the first portion towards the tip portion, the second portion defining an at least partially open section of the wall having a plurality of openings arranged to permit fluid flow into and out of the second portion of the body. The assembly further includes a hub attached to the proximal end of the catheter tube; and an introducer needle inserted through the catheter tube having an edge that projects beyond the distal end of the tube for penetrating a wall of the vessel.

Typically, the catheter assembly further includes a guidewire that extends through a lumen of the introducer needle for use in guiding the catheter tube into the vessel. This enables correct placement of the catheter within the vessel using a modified Seldinger technique. Alternatively, a guidewire may be introduced into the vessel through a separate catheter using the traditional Seldinger technique.

In the example illustrated in FIG. 2, the plurality of openings are elliptically shaped and substantially the same length whilst the length L₁ is approximately a third of the total length of the body and length L₂ is approximately two thirds the total length of the body.

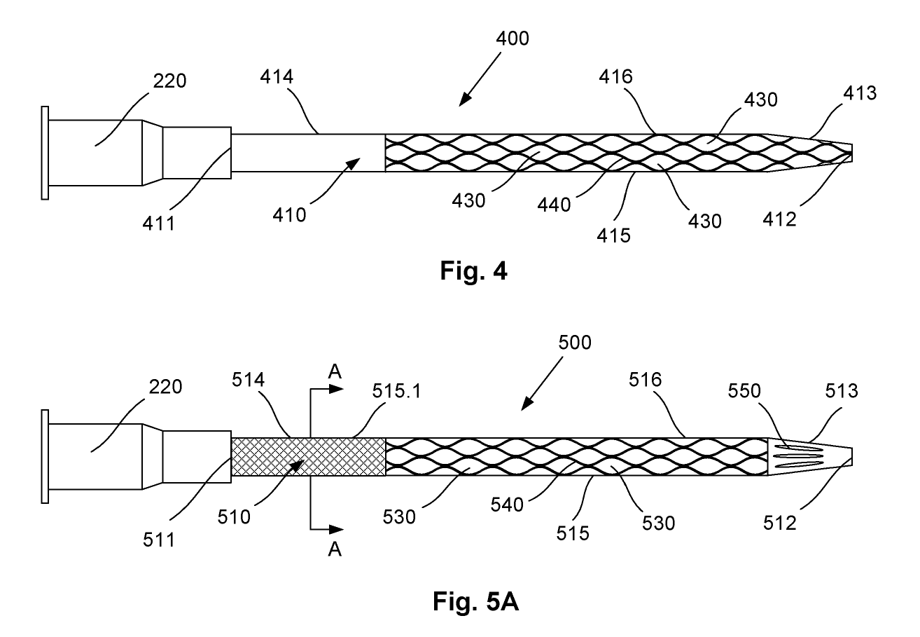

A second example of a catheter 300 for inserting into a vessel of a patient will now be described with reference to FIG. 3. The catheter 300 includes an elongate body 310 that extends from a proximate end 311 connectable to a hub 220 to a distal end 312 at which a fluid outlet is provided in a tip portion 313 thereof. The body 310 includes a first portion 314 that is an enclosed section and a second portion 316 that defines an at least partially open section having a plurality of openings 330, 332, 334 in a circumferential wall portion 315 thereof. In this example, the plurality of openings 330, 332, 334 are elliptically shaped and configured to extend in a direction of elongation of the body 310 and include openings of varying length.

In this example, at least one of the inner and/or outer surfaces of the circumferential wall 315 of the body 310 is textured or roughened (denoted by the cross-hatching) so as to reduce biofilm adherence to the body.

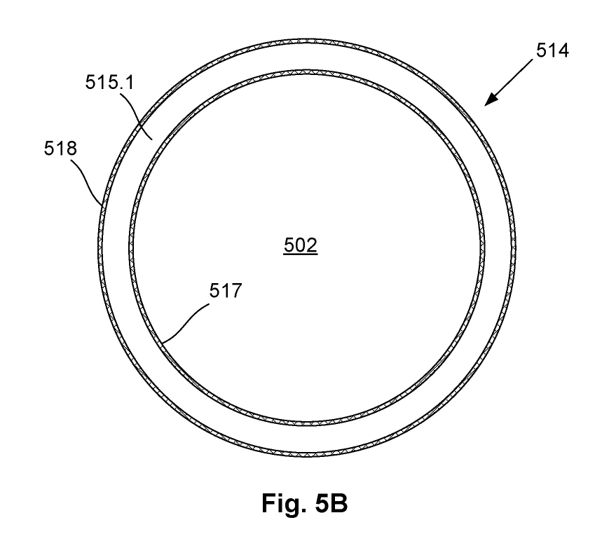

A third example of a catheter 400 for insertion into a vessel of a patient will now be described with reference to FIG. 4.

In this example, the catheter 400 includes an elongate body 410 that extends from a proximate end 411 connectable to a hub 220 to a distal end 412 at which a fluid outlet is provided in a tip portion 413 thereof. The body 410 includes a first portion 414 that is an enclosed section and a second portion 416 that defines an at least partially open section having a plurality of openings 430 in a circumferential wall portion 415 thereof. In this example, the plurality of openings 430 are arranged in a matrix or web-like formation. The circumferential wall 415 of the second portion 416 of the catheter body is formed in a web, mesh or lattice like structure 440 that may have a wave form or the like as shown. In this example, the second portion 416 includes the tip portion 413 so that the matrix or mesh extends to the distal end 412 of the catheter body 410.

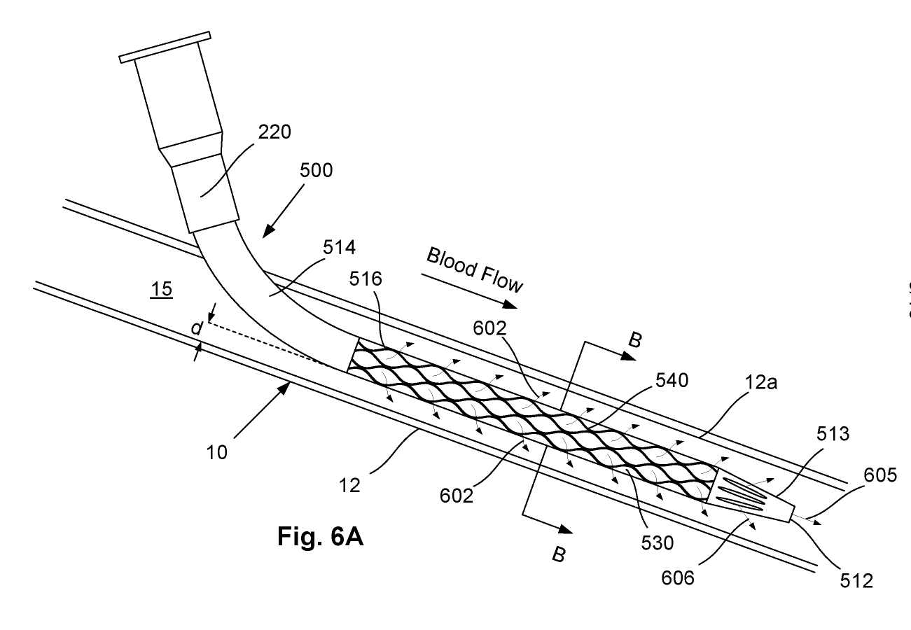

A fourth example of a catheter 500 for insertion into a vessel of a patient will now be described with reference to FIG. 5A.

In this example, the catheter 500 includes an elongate body 510 that extends from a proximate end 511 connectable to a hub 220 to a distal end 512 at which a fluid outlet is provided in a tip portion 513 thereof. The body 510 includes a first portion 514 that is an enclosed section and a second portion 516 that defines an at least partially open section having a plurality of openings 530 in a circumferential wall portion 515 thereof In this example, the plurality of openings 530 are arranged in a matrix or web-like formation. The circumferential wall 515 of the second portion 516 of the catheter body is formed in a web, mesh or lattice like structure 540 that may have a wave form or the like as shown. In this example, the second portion 516 terminates at the start of the tip portion 513 which is provided with separate elliptically shaped openings 550, although this is not essential and the tip portion could be solid.

In the example shown in FIG. 5A, the inner and outer circumferential wall of the first portion 514 is textured or roughened so as to reduce biofilm adhesion. As most clearly illustrated in FIG. 5B, both the inner surface 517 and outer surface 518 of the wall 515.1 of the first portion 514 have an applied micro-texture or roughness to reduce the smoothness of the polymer surfaces of the body.

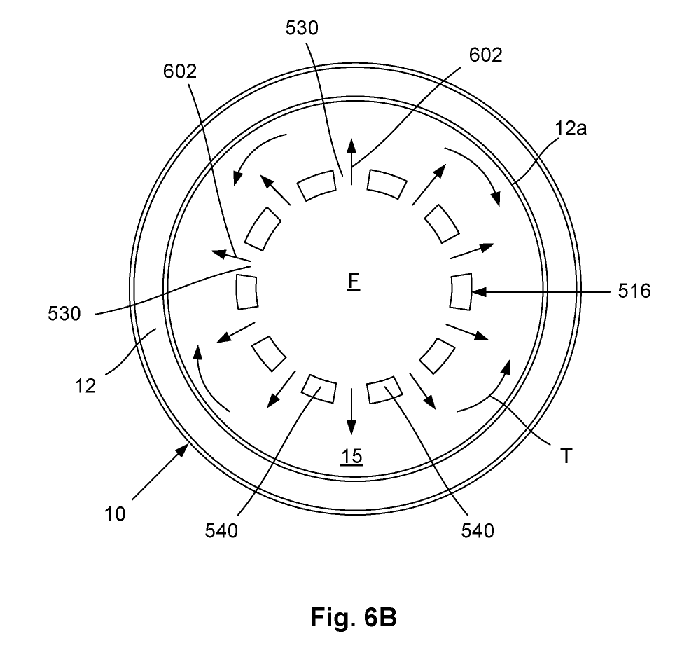

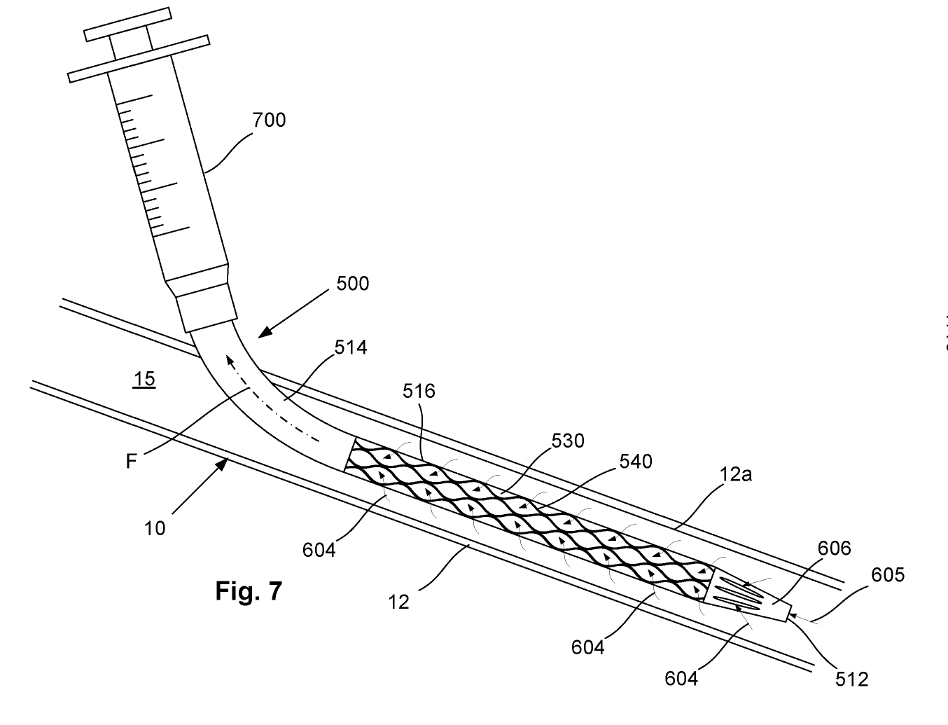

Example uses of the catheter 500 shall now be described with reference to FIGS. 6A, 6B and 7.

Referring firstly to FIGS. 6A and 6B, there is shown the catheter 500 inserted into a vessel 10 of a patient used to delivery a fluid such as an IV medicament, fluid, nutrition or blood component into the vessel. In use, the catheter 500 is centrally located within the vessel cavity 15 so as to be spaced apart from the inner surface 12a of the vessel wall 12 by a distance ‘d’. The spacing is achieved as a result of an equilibrium of forces acting on the catheter from the fluid outflow 602 from the plurality of openings 530 in the second portion 516 of the body 510. In the second portion 516, the fluid is permitted to exit the body uniformly around the circumference of the body. The fluid flow T around the catheter body thereby provides a ‘fluid cushioning’ effect which assists in urging the catheter away from the tunica intima 12a to thereby eliminate drag and concentration of outflow onto any singular point on the vessel wall. In this way, fluid is able to flow from the proximal end of the catheter body through the solid first portion 514 and exit the body through the plurality of openings 530. A portion of the fluid flow 606 may exit the body through apertures provided in the tip portion 513 and a further portion of the fluid flow 605 may still exit the body through the fluid outlet in the distal end 512.

Referring now to FIG. 7, the catheter 500 is shown inserted into a vessel 10 of a patient and connected to a syringe 700 for aspirating blood or fluid from the vessel 10 for sampling or the like. In this example, blood or other fluid F may be drawn into the catheter by way of inflow 604 through the plurality of openings 530 provided in the second portion 516 whereby it is sucked into the solid first portion 514 and drawn up into the syringe 700. A portion of the inflow 605, 606 may be through the fluid inlet at the distal end 512 of the body 510 or through openings provided in the tip portion 513 thereof respectively. This arrangement permits blood to be sampled more easily as even if there is an occlusion at the tip or if the catheter is sitting against the vein wall, blood can still flow into the catheter through the plurality of openings in the second portion 516 of the body 510.

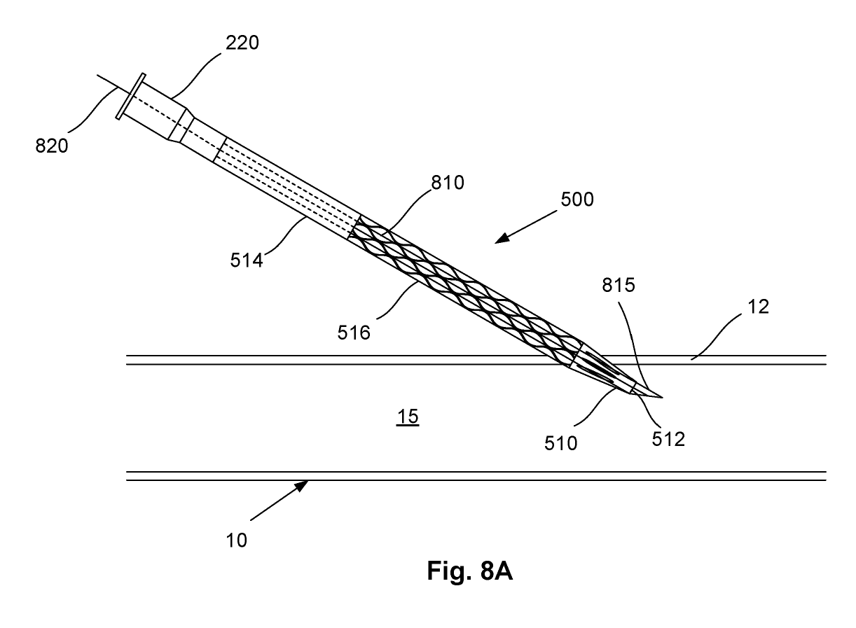

The above mentioned catheters are typically required to be inserted over a well placed guidewire within the vessel, using the traditional Seldinger or modified Seldinger technique (where the guidewire is deployed from within the insertion needle), to avoid subcutaneous extravasation of fluid. FIGS. 8A and 8B illustrate an example of insertion of the catheter 500 into the vessel 10 using the modified Seldinger technique.

In FIG. 8A, the catheter 500 is shown penetrating the vessel wall 12. The introducer needle 810 has a sharp bevelled end 815 which protrudes from the tip 512 of the catheter body 510. The bevel 815 pierces the vessel wall 12 allowing entry of the catheter tip. A guidewire 820 extends through the lumen of the needle 810. After the vessel wall 12 has been penetrated, the guidewire 820 is deployed through the needle 820 and into the vessel cavity 15. Once the guidewire 820 has been deployed inside the vessel 10, the needle 810 is pulled out at the same time as the catheter body 510 is inserted into the vessel 10. The catheter body 510 is guided into the vessel 10 over the guidewire 820 to ensure correct placement as shown in FIG. 8B. The guidewire 820 is then pulled out through the hub 220 thereby leaving the catheter 500 placed in the vessel 10 for subsequent use.

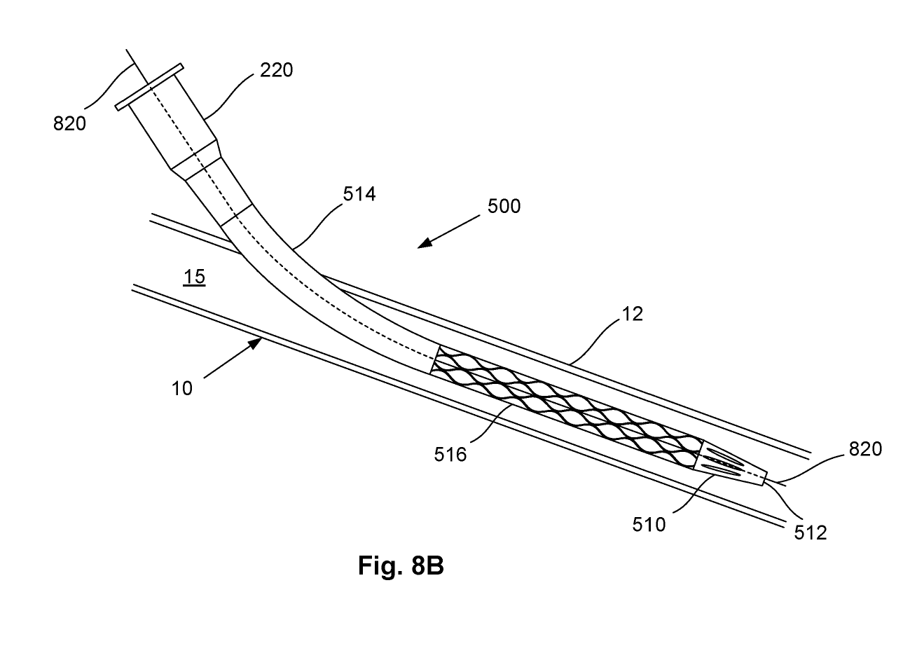

A fifth example of a catheter 900 for insertion into a vessel of a patient will now be described with reference to FIG. 9.

In this example, the catheter 900 includes an elongate body 910 that extends from a proximate end 911 connectable to a hub 220 to a distal end 912 at a tip portion 913 thereof. A fluid outlet may be provided in the tip portion 913 as per previous examples. The body 910 includes a first portion 914 that is an enclosed section and a second portion 916 that defines an at least partially open section having a plurality of openings 930 in a circumferential wall portion thereof.

In this case, a length of the first portion 914 is approximately two thirds of the total length of the body 910 whilst a combined length of the tip portion 513 and the second portion 916 is approximately a third of the total length of the body 910. However, it will be appreciated that the relative proportions of the first and second portions 914, 916 may vary to suit the particular application of the catheter as discussed above.

The plurality of openings 930 are arranged in a matrix or web-like formation, in which the circumferential wall of the second portion 916 of the catheter body is formed in a web, mesh or lattice like structure 940. Compared to the examples of FIGS. 4 and 5A, the web, mesh or lattice like structure 940 in this example includes relatively thicker elements separating the plurality of openings 930, whilst the openings 930 themselves are a relatively smaller and more closely arranged.

This finer web, mesh or lattice like structure 940 of this example can help to permit more even fluid flow out of the second portion 916 of the body 920 by avoiding excessive flow through upstream openings 930 which may be the case in a courser web, mesh or lattice like structure 540. It will be appreciated that a similar configuration of the web, mesh or lattice like structure 940 as depicted in FIG. 9 may be used in the previously described examples.

In this example, the second portion 916 terminates at the start of the tip portion 913 which is solid (i.e. non-porous) and tapered. Compared to previous examples, the example of FIG. 9 shows the option of a tip portion 913 which tapers to a narrower distal end 912. The tip portion 913 may be narrowed in this manner to create sufficient pressure for fluid to flow through the openings 930 in the second portion rather than simply flowing through the fluid outlet in the tip portion 913.

In the example shown in FIG. 9, the inner and outer circumferential wall of the first portion 914 is micro-textured or roughened (denoted by cross-hatching) so as to reduce biofilm adhesion, in a similar manner as described for FIGS. 5A and 5B. Furthermore, the solid tip portion 913 may also be micro-textured or roughened in this example.

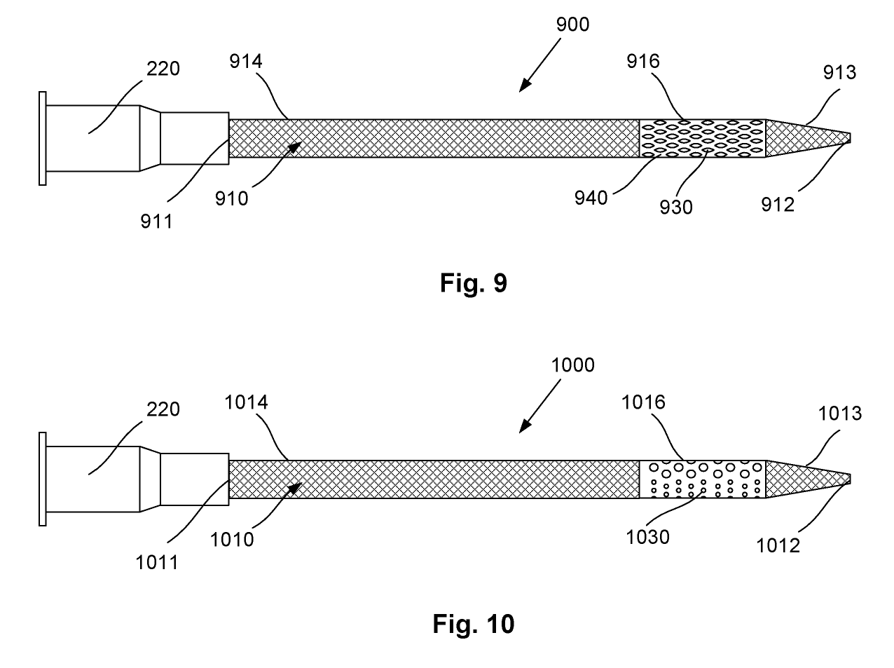

A sixth example of a catheter 1000 for insertion into a vessel of a patient will now be described with reference to FIG. 10. It will be appreciated that the example of FIG. 10 is generally similar to the example of FIG. 9, but for differences in the particular arrangements of the respective second portions.

In the example of FIG. 10, the catheter 1000 includes an elongate body 1010 that extends from a proximate end 1011 connectable to a hub 220 to a distal end 1012 at a tip portion 1013 thereof, in which a fluid outlet may also be provided. The body 1010 includes a first portion 1014 that is an enclosed section and a second portion 1016 that defines an at least partially open section having a plurality of openings 1030 in a circumferential wall portion thereof.

As per the previous example, the length of the first portion 1014 is approximately two thirds of the total length of the body 1010 whilst a combined length of the tip portion 1013 and the second portion 1016 is approximately a third of the total length of the body 1010, although the ratio of these lengths may vary depending on the intended use of the catheter 1000.

In this case, the openings 1030 are defined as round holes, although it should be understood that any suitable shape of openings 1030 may be used. The plurality of openings 1030 may be arranged in an array or any other repeating pattern with regular spacing between adjacent openings. The openings 1030 may have different sizes depending on their positioning on the catheter. In particular, in this example, plurality of openings 1030 includes a first arrangement of relatively larger holes across a first side of the second portion 1016 and a second arrangement of relatively smaller holes on an opposing second side of the second portion 1016.

In use, the catheter 1000 may be inserted into a vessel of a patient so that the first side having the relatively larger holes will be oriented upwardly and the second side having the relatively smaller holes will be oriented downwardly. In this manner, the different sizes of the holes may be used to account for variations in flow through the first and seconds sides under the influence of gravity. For instance, greater flow may be expected through the downwardly oriented (i.e. bottom) side of the inserted catheter 1000 compared to the upwardly oriented (i.e. top) side of the inserted catheter 1000, which could result in uneven flow around the circumference of the inserted catheter 1000 such that the inserted catheter 1000 may not be properly centralized within the vessel. However, this effect can be offset by the use of smaller holes on the bottom and large holes on the top of the inserted catheter 1000 to thereby change the relative flow proportions and thus assist in proper centralization of the inserted catheter 1000 within the vessel.

In some embodiments, the diameters of the holes on the first side of the second portion 1016 may be 1.5 to 2 times the diameters of the holes on the second side of the second portion 1016. In one particular implementation using a standard 20G catheter size (having an outer diameter of about 0.9 mm), the larger holes on the first side of the second portion 1016 may have a diameter of about 0.4 mm whilst the smaller holes on the second side of the second portion 1016 may have a diameter of about 0.25 mm. It should be understood that the sizes of the holes may vary depending on the gauge of the catheter 1000 and on the particular application.

In some examples more than two different sizes of holes may be used and arranged so that the holes progressively transition in size from the first side to the second side. These arrangements of different sized holes may have the same or different spacing between adjacent holes. It should be appreciated that similar variations in sizes may be applied to the configuration of openings arranged in a matrix or web-like formation as per previous examples.

As per the previous example, the second portion 1016 terminates at the start of the tip portion 1013 which is solid and narrowly tapered. The inner and outer circumferential wall of the first portion 1014 is micro-textured or roughened (denoted by cross-hatching), along with the solid tip portion 1013.

It should be understood that the features described above may be applied to catheters having various catheter gauge sizes and lengths and the sizing and proportions of particularly features may be selected based on the size of any given catheter and with regard to the intended use of the catheter. For instance, the second portion including the plurality of openings may be longer or shorter than indicated in the previous examples if required, whilst the size of the openings and the diameter of the fluid outlet in the tip portion may be varied depending on the catheter gauge size and other requirements.

It should also be understood that features depicted in the Figures are not necessarily shown to scale. For example, the relative sizes of particular features may be enlarged or reduced for the sake of improved understanding. Similarly, features may be represented in the Figures using simplified shapes or indicated using shading or the like to avoid unnecessarily complicating the views.

Accordingly, it will be appreciated that in at least one example the above described PIVC, midline or arterial catheter may eliminate or reduce catheter ‘drag’, occlusion, phlebitis, infiltration, biofilm adherence, shear force, outflow resistance and fibrin deposition.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

Persons skilled in the art will appreciate that numerous variations and modifications will become apparent. All such variations and modifications which become apparent to persons skilled in the art, should be considered to fall within the spirit and scope that the invention broadly appearing before described.

1. A catheter for insertion into a vessel of a patient, the catheter including an elongate body extending between a proximal end for attachment to a hub and a distal end at a tip portion thereof, the body having a circumferential wall and including a first portion that extends a first length from the proximal end and defines an enclosed section of the wall and a second portion that extends a second length from an end of the first portion to the tip portion, the second portion defining an at least partially open section of the wall having a plurality of openings arranged to permit fluid flow into and out of the second portion of the body, wherein the tip tapers to a narrower distal end and wherein the catheter is a peripheral catheter that is configured to be peripherally inserted into a peripheral vessel of a patient.

2. The catheter according to claim 1, wherein the plurality of openings are at least one of:
a) arranged in a matrix or web-like formation; and,
b) elliptically shaped and extend in a direction of elongation of the body.

3. (canceled)

4. The catheter according to claim 2, wherein the plurality of openings include:
a) openings having substantially the same length; and,
b) openings having varying length.

5. The catheter according to claim 1, wherein the plurality of openings include a first arrangement of openings on a first side of the second portion and a second arrangement of openings on an opposing second side of the second portion, the openings on the first side being larger than the openings on the second side.

6. The catheter according to claim 5, wherein a diameter of the openings on the first side is between 1.5 and 2 times a diameter of the openings on the second side.

7. The catheter according to claim 1, wherein the ratio of the first length to the second length is approximately in the range 0.5 to 2.

8. The catheter according to claim 1, wherein the plurality of openings reduce the surface area of the second portion of the body by at least one of:
a) between 30 and 40%;
b) between 40 and 50%;
c) between 50 and 60%;
d) between 60 and 70%; and,
e) between 70 and 80%.

9. The catheter according to claim 1, wherein the second portion includes the tip portion.

10. The catheter according to claim 1, wherein at least one of:
a) the circumferential wall defines an inner surface and an outer surface and wherein at least a portion of at least one of the inner and outer surface is textured or roughened so as to reduce biofilm adhesion to the body in use;
b) a surface of the tip portion is textured or roughened so as to reduce biofilm adhesion to the body in use;
c) an antibacterial coating is applied to at least a portion of the circumferential wall to reduce biofilm adhesion; and,
d) an antibacterial coating is applied to at least a portion of the circumferential wall to reduce biofilm adhesion, wherein the coating is an oil-infused polydimethylsiloxane (iPDMS).

11. (canceled)

12. (canceled)

13. (canceled)

14. The catheter according to claim 1, wherein in use, at least one of:
a) when the catheter is inserted into the vessel and fluid outflow is at a constant rate, forces acting on the body are such that a spacing is maintained between the body and an internal wall of the vessel; and,
b) at least a portion of the body is substantially centralized within the vessel.

15. (canceled)

16. A catheter for insertion into a vessel of a patient, the catheter including an elongate body extending between a proximal end for attachment to a hub and a distal end at a tip portion thereof, the body having a circumferential wall and including a first portion that extends a first length from the proximal end and defines an enclosed section of the wall and a second portion that extends a second length from an end of the first portion to the tip portion and defines an at least partially open section of the wall having a plurality of openings arranged to permit outflow of fluid from the body into the vessel such that in use, when the catheter is inserted into the vessel and fluid outflow is at a constant rate, forces acting on the body are such that a spacing is maintained between the body and an internal wall of the vessel.

17. The catheter according to claim 16, wherein the plurality of openings are arranged in a matrix or web-like formation.

18. The catheter according to claim 16, wherein at least a portion of the body is textured or roughened so as to reduce biofilm adhesion.

19. A catheter assembly, including:
a) a catheter tube for insertion into a vessel of a patient, the catheter tube having an elongate body extending between a proximal end and a distal end at a tip portion thereof, the body having a circumferential wall and including a first portion that extends a first length from the proximal end and defines an enclosed section of the wall and a second portion that extends a second length from an end of the first portion to the tip portion, the second portion defining an at least partially open section of the wall having a plurality of openings arranged to permit fluid flow into and out of the second portion of the body at multiple positions around a circumference, wherein the tip tapers to a narrower distal end and wherein the catheter is a peripheral catheter that is configured to be peripherally inserted into a vessel of a patient;
b) a hub attached to the proximal end of the catheter tube; and,
c) an introducer needle inserted through the catheter tube having an edge that projects beyond the distal end of the tube for penetrating a wall of the vessel.

20. The catheter assembly according to claim 19, further including a guidewire that extends through a lumen of the introducer needle for use in guiding the catheter tube into the vessel.

21. A catheter for insertion into a vessel of a patient, the catheter including an elongate body extending between a proximal end for attachment to a hub and a distal end at a tip portion thereof, the body having a circumferential wall and including a first portion that extends a first length from the proximal end and defines an enclosed section of the wall and a second portion that extends a second length from an end of the first portion to the tip portion, the second portion defining an at least partially open section of the wall having a plurality of openings arranged to permit fluid flow out of the second portion of the body, wherein the tip tapers to a narrower distal end, the catheter is a peripheral venous catheter that is configured to be peripherally inserted into a peripheral vein of a patient and wherein the catheter is inserted into the vessel and fluid outflow is at a constant rate, forces acting on the body are such that a spacing is maintained between the body and an internal wall of the vein.

22. A catheter for insertion into an artery of a patient, the catheter including an elongate body extending between a proximal end for attachment to a hub and a distal end at a tip portion thereof, the body having a circumferential wall and including a first portion that extends a first length from the proximal end and defines an enclosed section of the wall and a second portion that extends a second length from an end of the first portion to the tip portion, the second portion defining an at least partially open section of the wall having a plurality of openings arranged to permit fluid flow into the second portion of the body, wherein the tip tapers to a narrower distal end, the catheter is a peripheral arterial catheter that is configured to be peripherally inserted into a peripheral artery of a patient and when the catheter is inserted into the vessel fluid inflow can occur through holes along the second portion.

23. A catheter for insertion into a vessel of a patient, the catheter including an elongate body extending between a proximal end for attachment to a hub and a distal end at a tip portion thereof, the body having a circumferential wall and including a first portion that extends a first length from the proximal end and defines an enclosed section of the wall and a second portion that extends a second length from an end of the first portion towards the tip portion, the second portion defining an at least partially open section of the wall having a plurality of openings arranged to permit fluid flow into and out of the second portion of the body and wherein a ratio of the first length to the second length is approximately in the range 0.5 to 2.

24. A catheter for insertion into a vessel of a patient, the catheter including an elongate body extending between a proximal end for attachment to a hub and a distal end at a tip portion thereof, the body having a circumferential wall and including a first portion that extends a first length from the proximal end and defines an enclosed section of the wall and a second portion that extends a second length from an end of the first portion towards the tip portion, the second portion defining an at least partially open section of the wall having a plurality of openings arranged to permit fluid flow into and out of the second portion of the body and wherein the circumferential wall defines an inner surface and an outer surface and wherein at least a portion of at least one of the inner and outer surface is textured or roughened so as to reduce biofilm adhesion to the body in use.
